(19) United States
(12) Patent Application Publication
(10) Pub. No.: US 2014/0163389 A1
(43) Pub. Date: Jun. 12, 2014

(54) IN VIVO DETECTION OF EOSINOPHILS

(71) Applicant: The Arizona Board of Regents on Behalf of the University of Arizona, Tucson, AZ (US)

(72) Inventors: Michael W. Kudenov, Cary, NC (US); Bhaskar Banerjee, Tucson, AZ (US)

(73) Assignee: The Arizona Board of Regents on Behalf of the University of Arizona

(21) Appl. No.: 14/102,386

(22) Filed: Dec. 10, 2013

Related U.S. Application Data
(60) Provisional application No. 61/797,598, filed on Dec. 10, 2012.

(51) Int. Cl. A61B 5/00

(52) U.S. Cl. CPC A61B 5/0075; A61B 5/0071
USPC 600/476

(57) ABSTRACT

Snapshot spectral images viewing down the axis of the esophagus are processed to identify eosinophils. The snapshot images are based on fluorescence emitted in response to excitation optical radiation at two or more wavelengths. Ratio of spectral powers between snapshot images can be used in identification. In some examples, a relative abundance or density eosinophils is obtained, and processed to perform an in vivo assessment of tissue, such as esophageal tissue.

### CROSS REFERENCE TO RELATED APPLICATION

This application claims the benefit of U.S. Provisional Patent Application 61/797,598, filed Dec. 10, 2012, which is incorporated herein by reference.

### FIELD

The disclosure pertains to tissue assessment based on snapshot spectral images.

### BACKGROUND

Various inflammatory conditions exist which involve the accumulation of specific types of inflammatory cells in a localized area. For example, eosinophilic esophagitis (EoE) is an increasingly common allergic condition of the esophagus marked by an accumulation of specific inflammatory cells (eosinophils) that produces dysphagia (difficulty in swallowing), food impaction, persistent reflux symptoms in adults and failure to thrive in infants. EoE is currently diagnosed by endoscopy and biopsy. The cellular response is patchy and requires multiple (5 recommended) biopsies for diagnosis. The condition has been reported worldwide, with a prevalence of 1 in 2500 in Europe and North America. In some communities, the prevalence is doubling every 4 years. It is found in 10% of patients with dysphagia with a normal appearing esophagus on endoscopy.

EoE patients with dysphagia and food impaction and persistent reflux symptoms, as well as other symptoms including nausea, vomiting, chest pain, abdominal pain, food intolerance, failure to thrive, have biopsies taken from the esophagus for diagnosis. This requires endoscopy with sedation and five biopsies from the esophagus. Diagnosis is based on histopathology and usually takes 3-5 days. Biopsies entail risk, and as many patients present as emergencies, by the time biopsy results are available, it is too late to initiate therapy as many patients do not return after endoscopy. Some of these will present again which adds to the cost of care. Therefore, there is a need for rapid, point-of-care testing for the presence or absence of a clinical condition such as EoE that can involve the accumulation of specific inflammatory cells. There is also a need for rapid testing for the presence or absence of healthy tissue in a sample.

### SUMMARY

Systems for real-time in vivo imaging of a tissue sample region containing at least one autofluorescent cell include an excitation source configured to deliver excitation radiation to the tissue sample region at one or more excitation wavelengths. A snapshot spectral imager receives optical radiation emitted in response to the excitation radiation from at least one autofluorescent cell, and an image processor detects one or more target features in the tissue sample region based on the spectral images. In one example, the target features are autofluorescent cells such as eosinophils, and the image processor determines an estimate of a number of target features per target area in the tissue sample region. In other examples, the spectral imager produces esophageal images corresponding to a view along an esophageal axis. In some examples, the spectral imager can be turned to face the esophageal wall in a non-axial manner, to provide a more detailed view of a region of the esophagus. In yet other examples, the excitation source is configured to deliver excitation radiation to the tissue sample region at a first excitation wavelength and a second excitation wavelength, and the image processor detects the target feature based on ratios of received emitted optical power associated with the first excitation wavelength and the second excitation wavelength.

Methods of analyzing a tissue sample region containing at least one autofluorescent cell include irradiating the region at a plurality of excitation wavelengths and detecting emitted optical radiation generated in response to the excitation from the at least one autofluorescent cell at a plurality of emission wavelengths. A location of the at least one autofluorescent cell is determined based on the detected optical radiation at the plurality of emission wavelengths. In some examples, the emitted optical radiation is detected so as to form corresponding spectral images, and the location of the at least one autofluorescent cell is identified based on the spectral images. In one embodiment, the location of the at least one autofluorescent cell is identified based on ratios of received emitted optical radiation associated with the first excitation wavelength and the second excitation wavelength at the plurality of emission wavelengths. Typically, the emitted optical radiation from the at least one autofluorescent cell is detected by snapshot imaging so as to form spectral images based on emitted optical radiation associated with the first and second excitation wavelengths. In a specific application, the target region is a portion of an esophagus, and the snapshot images are images viewing along an axis of the esophagus. In still other examples, an image of the target region is displayed that includes an indication of a clinical level associated with the density of the plurality of autofluorescent cells. In some examples, the clinical level is dependent on axial location in the esophagus.

The foregoing and other objects, features, and advantages will become more apparent from the following detailed description, which proceeds with reference to the accompanying figures.

### BRIEF DESCRIPTION OF THE DRAWINGS

FIG. 1 is a schematic diagram of an exemplary endoscope system configured for hyperspectral detection of fluorescence and production of specimen images based on the detected fluorescence.

FIGS. 2A-2B illustrate a spectral imager configured to process spectral images based on an eosinophil emission spectrum.

FIG. 3 is a schematic diagram of a SHIFT spectrometer situated to image a tissue specimen.

FIG. 4 is a schematic diagram of a SHIFT spectrometer configured to receive an image from a coherent fiber bundle.

FIG. 5 is a schematic diagram of an esophageal probe that includes a spectral imager that is insertable into the esophagus.

FIG. 6A illustrates a specimen image that displays eosinophil counts.

FIG. 6B illustrates spectral splices in a specimen image.

FIG. 7 illustrates a representative method of assessing tissue.

FIG. 8 is a chart showing eosinophil excitation and emission spectra. An emission spectrum produced for 450 nm excitation is shown as a solid line, while an emission spectrum produced for 400 nm excitation is shown as a dashed line. The excitation and emission spectra illustrate ranges of wavelengths that are suitable for excitation and detection, respectively.

FIG. 9 illustrates a portion of a probe.

FIG. 10 illustrates a system configured to acquire and process spectral images for tissue assessment.

FIG. 11 is a flow chart showing an exemplary method for detecting the presence or absence of autofluorescent cells or tissue to aid in the diagnosis of a clinical condition and/or the treatment of a subject.

FIG. 12 illustrates a representative feed-forward neural network for tissue assessment based on principal components.

FIGS. 13A-13B are photographs showing linear component analysis processed fluorescence images showing microsphere spectral correlation and background correlation for a high concentration of microspheres, respectively.

FIGS. 14A-14B are photographs showing linear component analysis processed fluorescence images showing microsphere spectral correlation and background correlation for a low concentration of microspheres, respectively.

FIG. 15 illustrates spectral imaging along an axis of an esophagus so as to perform tissue assessment.

FIG. 16 is a schematic diagram of an exemplary computing environment associated with a hyperspectral detection system.

FIGS. 17A-17D are representative esophageal images showing eosinophil counts.

### DETAILED DESCRIPTION

As used in this application and in the claims, the singular forms “a,” “an,” and “the” include the plural forms unless the context clearly dictates otherwise. Additionally, the term “includes” means “comprises.” Further, the term “coupled” does not exclude the presence of intermediate elements between the coupled items.

The systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved. Any theories of operation are to facilitate explanation, but the disclosed systems, methods, and apparatus are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

In some examples, values, procedures, or apparatus' are referred to as “lowest”, “best”, “minimum,” or the like. It will be appreciated that such descriptions are intended to indicate that a selection among many used functional alternatives can be made, and such selections need not be better, smaller, or otherwise preferable to other selections.

For convenience in the following description, the terms “light” and “optical radiation” refer to propagating electromagnetic radiation that is received from one or more objects to be imaged or otherwise investigated. As used herein, an optical flux refers to electromagnetic radiation in a wavelength range of from about 100 nm to about 100 μm. In some examples, an optical flux has a spectral width that can be as large as 0.5, 1, 2, 5, or 10 times a center wavelength, or can comprises a plurality of spectral components extending over similar spectral bandwidths. Such optical fluxes can be referred to as large bandwidth optical fluxes. A visible optical flux generally has a spectral bandwidth between about 400 nm and 700 nm. In some examples discussed below, optical fluxes are associated with fluorescence spectra. Typically, an optical flux is received from a scene of interest and amplitude, phase, spectral, or polarization modulation (or one or more combinations thereof) in the received optical flux is processed based on a detected image associated with a spatial variation of the optical flux which can be stored in one or more computer-readable media as an image file in a JPEG or other format. In the disclosed examples, so-called “snapshot” imaging systems are described in which image data associated with a plurality of regions or locations in a scene of interest (typically an entire two dimensional image) can be obtained in a single acquisition of a received optical flux using a two dimensional detector array. However, images can also be obtained using one dimensional arrays or one or more individual detectors and suitable scanning systems. In some examples, an image associated with the detected optical flux is stored for processing based on computer executable instruction stored in a computer readable medium and configured for execution on general purpose or special purpose processor, or dedicated processing hardware. In addition to snapshot imaging, sequential measurements can also be used. For convenience, examples that provide two dimensional images are described, but in other examples, one dimensional (line) images or single point images can be obtained.

For convenience, optical systems are described with respect to an axis along which optical fluxes propagate and along which optical components are situated. Such an axis can be shown as bent or folded by reflective optical elements. In the disclosed embodiments, an xyz-coordinate system is used in which a direction of propagation is along a z-axis (which may vary due to folding of the axis) and x- and y-axes define transverse planes. Typically the z-axis is in the plane of the drawings and defines a system optical axis. In other examples, lens arrays are used to produce a plurality of images of an object. In some examples, such images are referred to as sub-images and are associated with sub-image optical fluxes.

### DEFINITIONS

Autofluorescence: Fluorescence emitted by an autofluorescent compound or cell, such as an eosinophil. Of particular interest herein are those native fluorophores that exhibit an association with inflammation. These native fluorophores exhibit an increased or decreased fluorescence in association with an inflammatory process occurring in the vicinity of the fluorophore. Such an association may reflect an underlying positive or negative correlation with the inflammatory process, such as increased or decreased abundance and/or bioactivity of the fluorophore (such as increased abundance of eosinophils in EoE).

Hyperspectral Image: A hyperspectral image typically contains image data for a plurality of image locations as a function of wavelength, and can be represented as a three dimensional array. Any of a number of different techniques may be used to produce a hyperspectral image or hyperspectral data, including scanning an image spatially, capturing full spectral data sequentially; scanning an image spectrally, capturing full spatial information sequentially, and taking a “snapshot” (capturing all the spectral and spatial information in a single data acquisition). Spectral data can be associated with visible or other wavelength regions.

Real-time: The performance of an imaging or analysis step (such as data analysis, image production, or spectra comparison) substantially simultaneous to the acquisition of the underlying data. Thus, real-time imaging can refer to the production of an image of a tissue region that occurs a relatively short period of time following the acquiring of the first piece of physical data from the sample.

Diagnosis: Identifying the presence or nature of a biological or medical condition, such as, but not limited to, presence of a mutation, or systemic or localized concentration in a subject of a particular inflammatory cell or particular pathological or healthy tissue type.

### Introduction

Hyperspectral detection systems are disclosed herein for the detection of particular histological conditions which involve the accumulation of autofluorescent cells. The disclosed hyperspectral imaging systems may be capable of tunable spectral resolution and may be configured to provide real-time data, such as real-time images. The system can be compact and may use small-format cameras, such that the device could enable in vivo low light hyperspectral endoscopy, including video endoscopy. In one embodiment, the hyperspectral imaging system is a hyperspectral pill camera that can be ingested. The system can comprise a sensor employing a polarization grating, which can enable electro-optically tunable spectral resolution. In one embodiment, the sensor can specifically convert raw data into processed spectral output in about 200 ms.

In one aspect, one or more components of the hyperspectral detection system (such as the entire detection system) can be passed through an interior of an endoscope. In various embodiments, the system can comprise a disposable or non-disposable fiber optic probe. The probe can be specifically passed through the biopsy channel of a standard endoscope, such as for the real-time detection of clinical conditions of the esophagus and/or other organs. In other embodiments, the probe can be passed into a body lumen independent of an endoscope. In various implementations, the system can accurately detect an inflammatory and/or allergic condition.

In another example, the disclosed systems can detect eosinophilia in a tissue sample in vivo, which may assist in the diagnosis of conditions such as EoE, asthma, allergic rhinitis, and eosinophilic conditions of the skin and eye. Eosinophils display a particular autofluorescence pattern due to the presence of a large number of granules in its cytoplasm that contain flavin adenine dinucleotide (FAD). Thus, in various embodiments, the disclosed systems can exploit the unique autofluorescence spectrum of eosinophils due to FAD. In one embodiment, a plurality of fluorescence wavelengths that includes optical radiation between about 480 nm-600 nm, 500 nm-550 nm, or 500 nm-520 nm can be used to detect the presence or absence of eosinophils. By detecting eosinophils in real-time, a user can perform one or more of the following: (1) reduce the need for biopsies and histological diagnosis; (2) prevent delay in initiating treatment; (3) enable monitoring of response to therapy; and/or (4) diagnose recurrence. In various embodiments, diagnosis of eosinophilia is coupled with administration of a treatment or other intervention which may include administration of a proton pump inhibitor or steroid, a dietary change and/or evaluation for a food allergy.

Eosinophils are noted for marked autofluorescence (AF) emission at 520 nm, which exceeds other cells including leukocytes, due to the abundance of a large number of cytoplasmic granules that contain FAD. Although other tissue constituents such as collagen, elastin, and other cellular flavoproteins also fluoresce at 520 nm, the increased fluorescence from the cytoplasmic granules permits identification of clusters of eosinophils using the disclosed methods and apparatus.

In some embodiments, the disclosed systems define one or more diagnostic criteria and/or algorithms. The criteria and/or algorithms to be applied may be stored in a location within the local computing environment and/or may be accessible via a network connection. For example, a demonstration of 15 eosinophils per high power field (HPF) can be a diagnostic criteria or input for a diagnostic algorithm for EoE. In other examples, the disclosed systems can be arranged to compare isolated spectrum of autofluorescent cell(s) of interest to pre-collected spectral data (e.g., “training data”) contained within a library that correspond to one or more histological and/or clinical conditions, such as eosinophilia. Training data can be used in conjunction with a neural network to enhance image contrast.

Some disclosed embodiments are directed to the detection, evaluation, and treatment of eosinophilic disease. Eosinophilic diseases can involve multiple organs including the esophagus, stomach, intestines, lungs, naso-pharynx and skin. Increased numbers of eosinophils cannot be seen by the naked eye and their patchy distribution requires multiple biopsies, which is time consuming, expensive and open to sampling error. As disclosed herein, spectral mage based real-time detection of eosinophils enables point-of-care diagnosis and prompt treatment.

Detecting of eosinophils can be challenging due to a cluttered background. The disclosed methods and apparatus use hyperspectral imaging to acquire continuous spectra along with image processing based on linear unmixing, principal component analysis, endmember analysis, and/or neural networks to aid in automated identification, or to provide enhanced images for clinician viewing. In addition, the disclosed methods and apparatus reduce measurement acquisition times, leading to increased patient comfort and reducing costs. Data acquisition time is primarily limited by temporal, spatial, or spectral scanning (e.g., time multiplexing) and acquisition of diagnostically relevant optical data in a wide-field and high-throughput (snapshot) imaging modality can reduce acquisition time. Accordingly, spatial multiplexing of spectral and spatial data is used, without temporal multiplexing.

The disclosed methods and apparatus can be applied to any eosinophil-related disease in tissues, including the skin, esophagus, naso-pharynx, lungs, stomach and intestines to identify spectral signatures of eosinophils without contacting tissues of interest. Thus, an “optical biopsy” is produced that can detect the presence and location of eosinophils without the cost and time-delay associated with standard histology. Results can be available within minutes or seconds. Snapshot image acquisition in which spectral data is acquired in a single integration time of the camera or in a single exposure also avoids problems associated with patient movement.

An image based optical diagnostic probe as disclosed herein can be inserted into the esophagus without using an endoscope, in an un-sedated patient, for real-time diagnosis of esophageal eosinophils. This would allow point-of-care real-time diagnosis in a variety of non-specialized settings, without endoscopy or sedation such as at a doctor's office, urgent care facility, nursing home, etc. The disclosed methods and apparatus can be used in ex vivo applications in which ex vivo tissue specimens from biopsies are evaluated based on fluorescence spectra.

Eosinophil distribution tends to be patchy, with clusters of eosinophils that require multiple biopsies to prevent false negative results, yielding an incorrect diagnosis. Using an optical spectral imaging sensor, sampling error can be eliminated. If tissue biopsy is necessary, spectral-image guided biopsy would also eliminate sampling error and maximize histologic yield (tissue biopsy can be directed to areas of high fluorescence intensity, rather than random spatial locations).

Response to therapy often requires tissue sampling and histology, which is expensive. Image-based optical testing, that confirms or excludes the presence of eosinophils, will reduce cost, and allow therapeutic changes to be made at the point-of-care, without delay.

Eosinophilic disease is not based on the presence of eosinophils alone, but increased concentration of eosinophils, represented by the number of eosinophils per high power field. However, the actual eosinophil counts (per high power field) depend on the field of view of the microscope (microscopes have different fields of view) used as well as the area of tissue biopsied. This leads to errors. Autofluorescence intensity can predict the concentration of eosinophils and thus provide an estimate of the degree of eosinophilia, as opposed to simple presence or absence. In addition, an imaging probe can have a well-defined field of view, eliminating or reducing field of view variations.

Microscopic determination of eosinophil counts per high power field requires a pathologist to ‘count’ eosinophils, which is time-consuming, requires an expert, and is prone to errors. Spectral imaging of tissue samples as disclosed can produce a real-time (ex-vivo) eosinophil count without a microscope, tissue preparation/staining, or an expert pathologist.

Accessing internal organs for imaging of eosinophils is simplified using an optical probe that can be passed independently into a lumen, over a guidewire, or through a naso-gastric tube. Such as system can be incorporated into a standard endoscope or a capsule endoscope.

Numerous examples of the disclosed technology are described below.

### Example 1

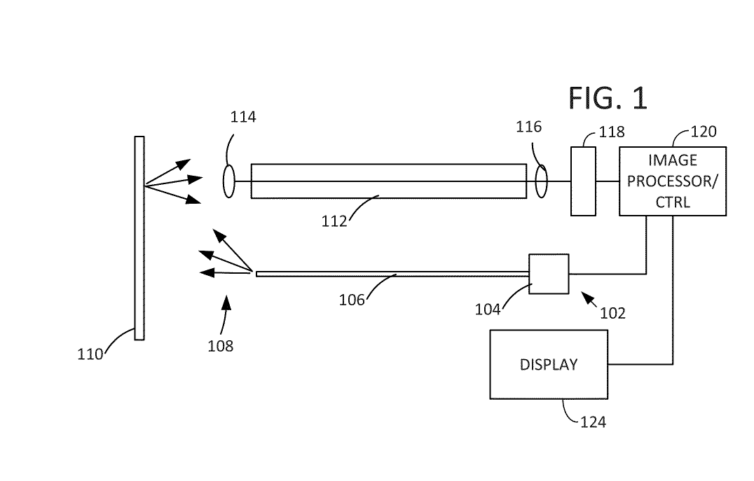

Referring to FIG. 1, a representative endoscope includes a fluorescence stimulation system 102 that includes an optical radiation source 104 that is coupled to a beam delivery optical fiber 106 so as to direct an excitation optical beam 108 to a specimen under investigation 110. The optical radiation source 104 can include one or more lasers, light emitting diodes, arc lamps, or other sources that can provide optical radiation at a suitable wavelength to stimulate fluorescence at the specimen. In typical applications, optical radiation at wavelengths between 400 nm and 500 nm is used. Narrow band irradiation such as laser radiation or broadband radiation such as produced by lasers, light emitting diodes, or other sources can be used. The optical radiation source 104 also includes a power monitoring system such as a photodiode and associated electronics that permits determination of excitation power delivered to the specimen 110.

A coherent fiber bundle 112 and lenses 114, 116 are situated to produce an image of a portion of the specimen 110 based on fluorescence from the specimen 110. A portion of the fluorescence is directed to a snapshot imager 118 that produces a specimen image as a function of radiation wavelength. In some examples, such images are represented as a three dimensional array defined by a two dimensional array of specimen locations and a one dimensional array of wavelengths. Thus, any particular image location can be associated with at least one specimen location and fluorescence at a plurality of wavelengths. These multi-wavelength images can be processed with an image processor 120 so as to enhance image contrast associated with a selected specimen constituent. For example, eosinophils can be emphasized (or deemphasized) in an image. A processed image can be directed to a display 124 for viewing by a clinician. Typically, processed or unprocessed images are stored as well for subsequent analysis and viewing.

The image processor 120 can also be arranged to control acquisition and analysis of images. For example, an excitation wavelength and/or power can be selected so that images associated with one or more different excitation beams can be acquired, and images processed based on a variety of potential specimen constituents of interest.

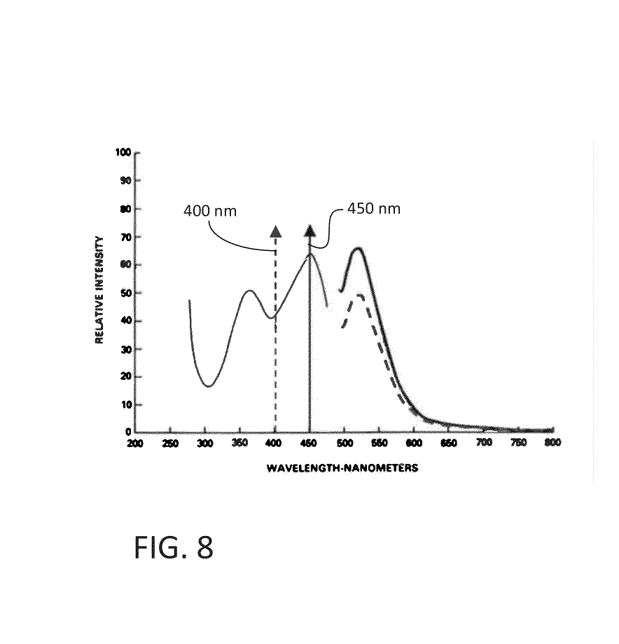

FIG. 8 illustrates an absorption spectrum and two fluorescence emission spectra associated with narrowband irradiation at about 400 nm and 450 nm. Fluorescence associated with 400 nm excitation is shown as a dashed line; fluorescence associated with 450 nm excitation is shown as a solid line. While the overall spectrum is substantially the same, fluorescence power per wavelength is less for 400 nm excitation. In some cases, some specimen features lack this power variation so that measurement of emitted power as a function of excitation power and wavelength permits identification of target cells, and enhancement of target cell visibility in images.

### Example 2

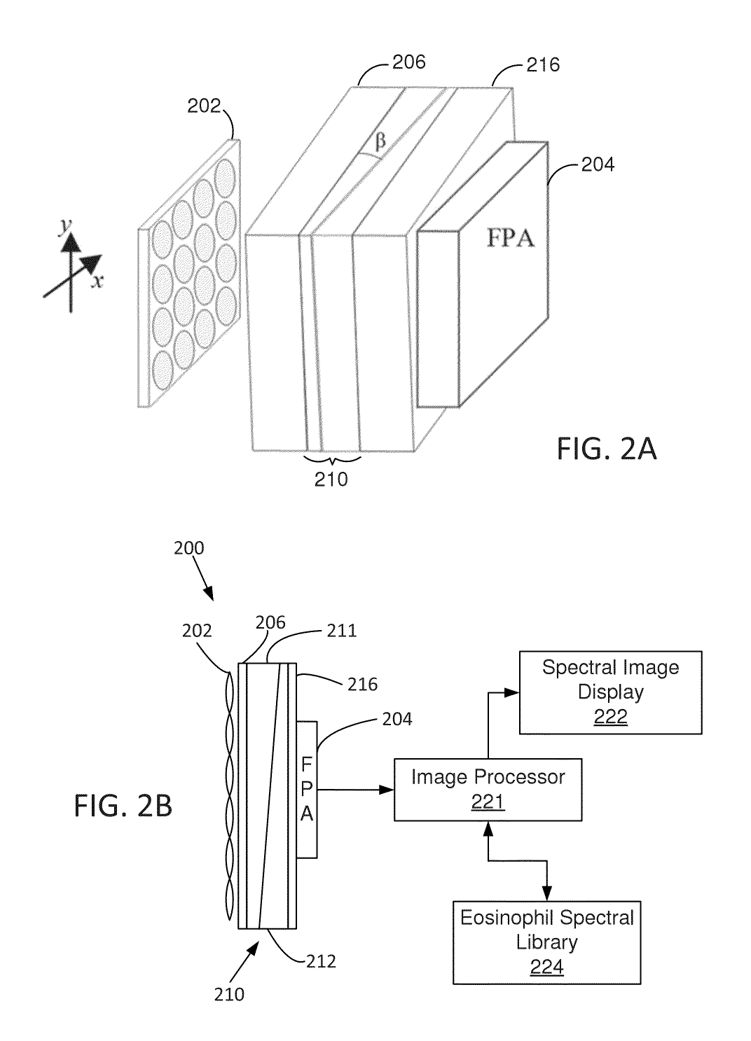

With reference to FIGS. 2A-2B, a representative imaging spectral interferometer 200 includes a lenslet array 202 that includes N by M lenses arranged in a rectangular array. The lenses of the array 202 form corresponding images of an object and direct the images to a focal plane array 204. The images are directed through a first polarizer 206, a birefringent prism pair 210, and a second polarizer 216. The prism pair has eigenpolarization states oriented at an angle δ degrees with respect to the x axis. The first polarizer 206 and the second polarizer 216 are linear polarizers having transmission axes that are tilted with respect to an x-axis toward a positive y-axis by an angle of 45+δ degrees. In this example, the sub-images formed by the lenslet array 202 include a polarization based optical path different (OPD) that is a function of the x-coordinate due the varying thickness of wedge prisms 211, 212 and that can produce interference.

An image processor 221 is coupled to the FPA 204 to receive electrical signals associated with optical interference caused by the OPD produced by the prism pair 210. The electrical image signals associated with one or all of the lenslets of the array 202 can be recorded, and combined with other recorded signals. Typically, the recorded signals are processed to obtain an image so as to form an interference map as a function of OPD and then Fourier transformed by the image processor 221. Spectral characteristics (emission and excitation spectra for eosinophils or other cells or tissues) are stored in a memory 222 as a spectral library. In some cases, measured spectral images of test specimens are stored for use in producing training sets for processing of images in clinical settings. A resulting spectral image is presented for visual inspection on a display 224, but additional prism pairs can be used to provide OPD variation along both x- and y-axes.

Images produced with the imaging spectral interferometer 200 include spectral power at a plurality of specimen locations for a plurality of wavelengths. Typically, spectral power is obtained at a very large number of wavelengths, 10-1000 wavelengths over a detection bandwidth of 20 nm, 50 nm, 100 nm, 200 nm, 300 nm or more. Displayed or stored images thus can be arranged as array of X by Y pixels, each pixel associated with a plurality of spectral powers. Image processing as discussed further below can be based on one or more spectral slices of such images, wherein one or more spectral planes or ranges of spectral planes are selected for analysis.

### Example 3

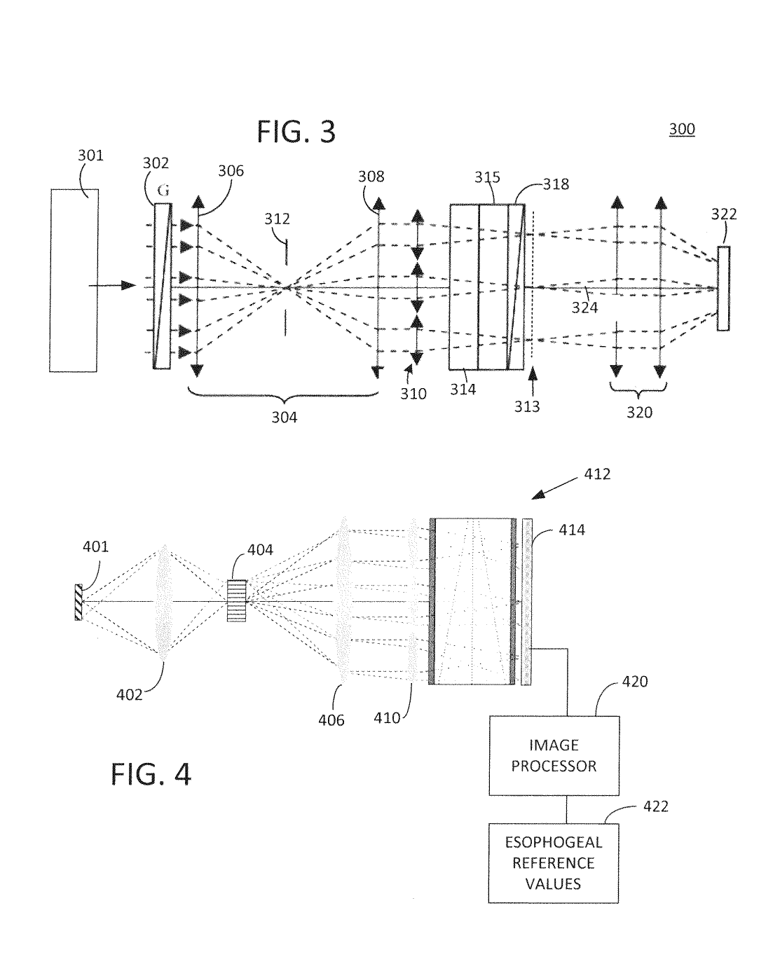

With reference to FIG. 3, a representative snapshot imaging Fourier transform imager 300 includes a linear polarizer 302 situated to receive an optical flux from an endoscope 301. A 1:1 afocal telescope 304 that includes an input lens 306 and an output lens 308 is situated to receive the optical flux from the polarizer 302 and deliver the optical flux to a lens array 310, such as a 10 by 10 array of lenses. A field stop 312 is situated at a focus of the input lens 306. Lenslets of the lens array 310 form respective images of the object and deliver the images to an intermediate image plane 313 through birefringent prism pairs 314, 315 and a linear polarization analyzer 318 that is re-imaged by relay optics 320 to a focal plane array 322. The prism pairs 314, 315 are situated to produce variable OPDs along orthogonal axes that are also orthogonal to a spectrometer axis 324.

In the example of FIG. 3, the afocal telescope 304 and the field stop 312 permit the images formed by the lenslets of the lens array 310 to be separated at the focal plane array 322. The relay optics 320 permit the image plane 313 of the lens array 310 to be re-imaged as needed. For a more compact instrument, the image plane 313 can be at the focal plane array 320, without relay optics. For convenient illustration, processing of the images detected by the focal plane array is not described in detail, but is based on Fourier transforms and the variable OPD provided by the prism pairs 314, 315. Additional details of such spectral analysis systems can be found in Kudenov, U.S. Patent Application Publication 20120268745, which is incorporated herein by reference.

### Example 4

Referring to FIG. 4, a spectral imaging arrangement suitable for use in endoscopy with a coherent fiber bundle includes an objective lens 402 situated to produce an image of a tissue region 401 (such as a portion of an esophagus or a tissue sample at the sample plane of a microscope) at an entrance surface of a coherent fiber bundle 404. A collimator 406 receives the image flux from the coherent fiber bundle 404 and directs the image flux to a lenslet array 410 and a two dimensional birefringent interferometer 412. An array detector 414 or camera receives the image flux and provides an interferometric image to an image processor 420 that determines power as a function of wavelength for some or all detector elements of the array detector. In some cases, only certain detector elements are used or provide independent values. In the example of FIG. 4, characteristic values associated with esophageal specimens are stored in a memory 422 for use in additional processing.

### Example 5

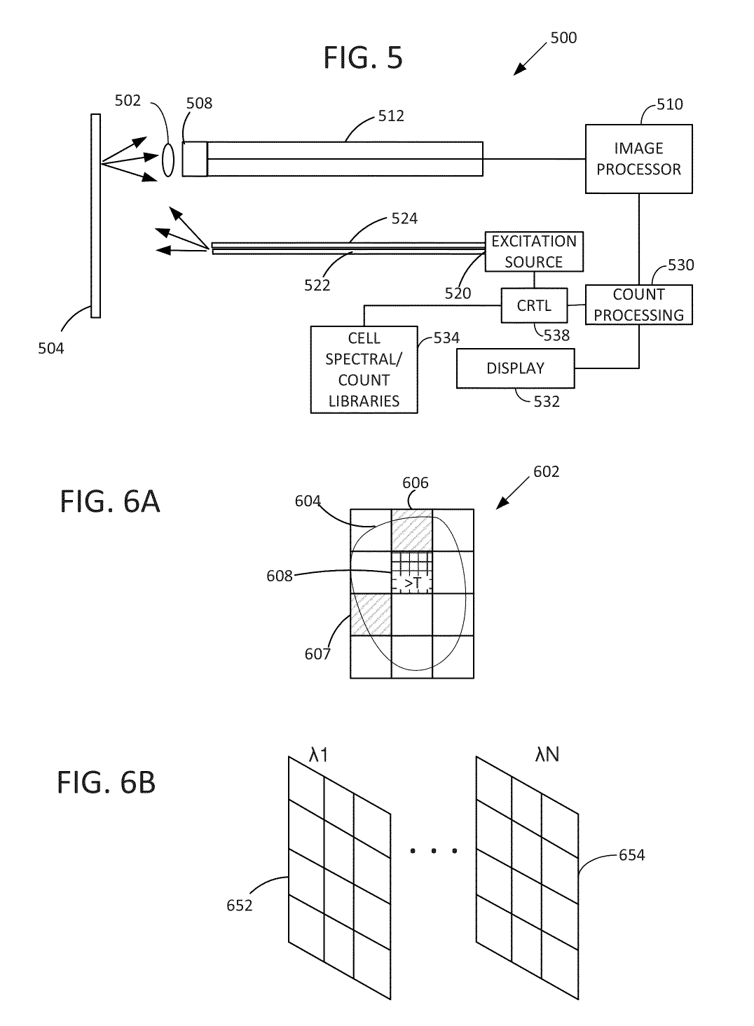

With reference to FIG. 5, a system 500 for in vivo tissue evaluations includes an objective lens 502 situated to direct an emitted optical flux from a tissue region 504 so as to form an image of the tissue region at a snapshot spectral imager 508. The snapshot spectral imager 508 produces an electrical signal associated with the image that is coupled to an image processor 510 through an endoscope tube 512. An excitation source 520 is coupled to one or more optical fibers 522, 524 that direct one or more excitation fluxes to the target region 504. In addition, a visible flux can be provided for direct imaging of the tissue region 504. The fibers 522, 524 can be included within the endo scope tube 512, but are shown separately for convenient illustration. Additional structures needed for treatment, tissue sampling, irrigation, or other procedures can also be included so that further steps in diagnosis and treatment can be performed based on acquired images.

The image processor 510 is generally configured to produce spectral images at a plurality of wavelengths based on, for example, a Fourier transform of a fringe pattern produced by a spectral imager that includes a birefringent interferometer. The spectral image is processed by a counting system 530 that determines an approximate count of target cells (such as eosinophils) at a plurality of tissue locations (at corresponding image pixels) based on the spectral images. One or more spectral images showing eosinophil emissions along with an indication of location eosinophil count is coupled to a display 532 for clinician inspection. As displayed, brightness variations can be associated with eosinophils so that eosinophil density in a target region can be evaluated. In addition, one or more or all pixels or selected pixel regions can be pseudocolor encoded to indicate normal eosinophil densities (for example, as green display regions) or abnormal eosinophil densities (for example, as red display regions). Tissue data associated with normal and abnormal values can be stored in memory 534 and a controller 538 is configured to coordinate target irradiation, data acquisition, image processing, cell counting, and display.

Selected properties of images produced as described above are illustrated in FIG. 6A. A target feature 604 in an image 602 is defined by a plurality of pixels, shown in FIG. 6A as 4 rows by 3 columns. Pixels 606, 607 have a shading associated with an intermediate value of an eosinophil count, and pixel 608 has a shading associated with a large value of an eosinophil count, such as value indicative of disease or indicative of a need for further investigation. In some cases, sets of pixels are shaded in this manner, and pixels 606, 607, 608 can also be viewed as sets of pixels associated with particular eosinophil counts. The pixel 608 also includes a numerical expression or value associated with the eosinophil count density. As shown in FIG. 6B, the image 602 includes image data at a plurality of wavelengths, shown as image slices 652, 654 at wavelengths λ1 and λN, respectively. Data in adjacent image slices can be spectrally independent, depending on spectral resolution, but need not be.

### Example 6

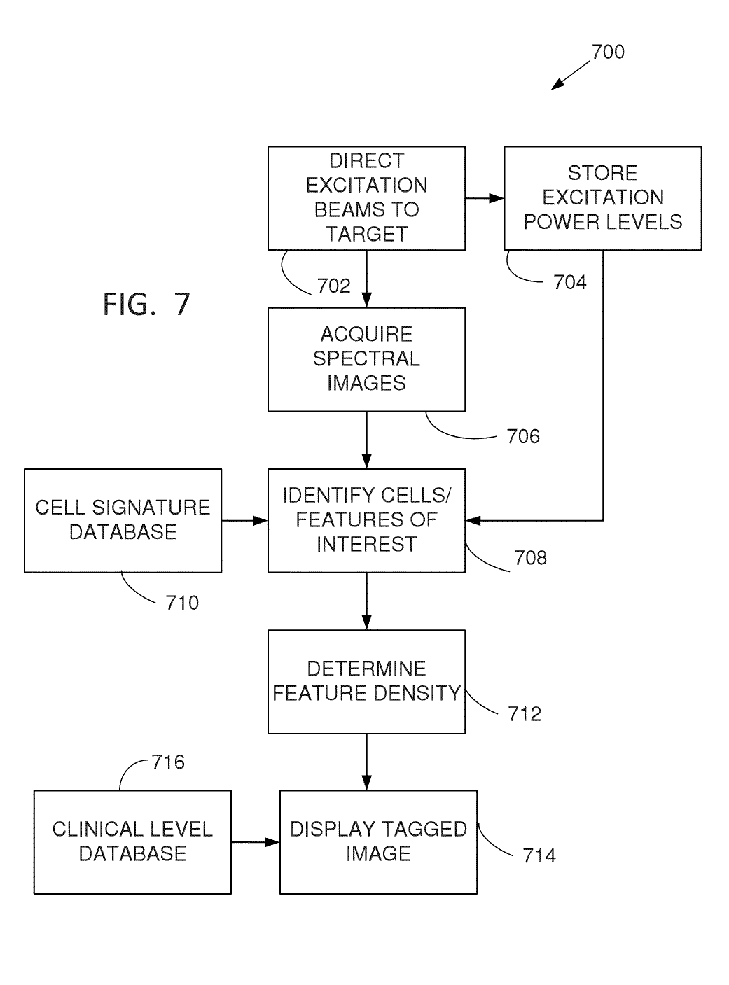

Referring to FIG. 7, a representative method 700 includes directing one or more excitation beams to a target at 702. Optical radiation emitted in response to the excitation beams is used to obtain spectral images at a plurality of wavelengths (or wavelength bands) at 706. Excitation power levels are stored at 704. At 708, cells or other features of interest are identified based on a cell/feature database 710. In some examples, eosinophils or other target species are distinguished based on emitted power as a function of excitation beam power and spectra stored in the database 710. At 712, a feature density (features/unit area) can be estimated and images tagged with the feature densities displayed at 714. A clinical level database 716 can also be used to customize images to indicate clinically significant feature densities.

### Example 7

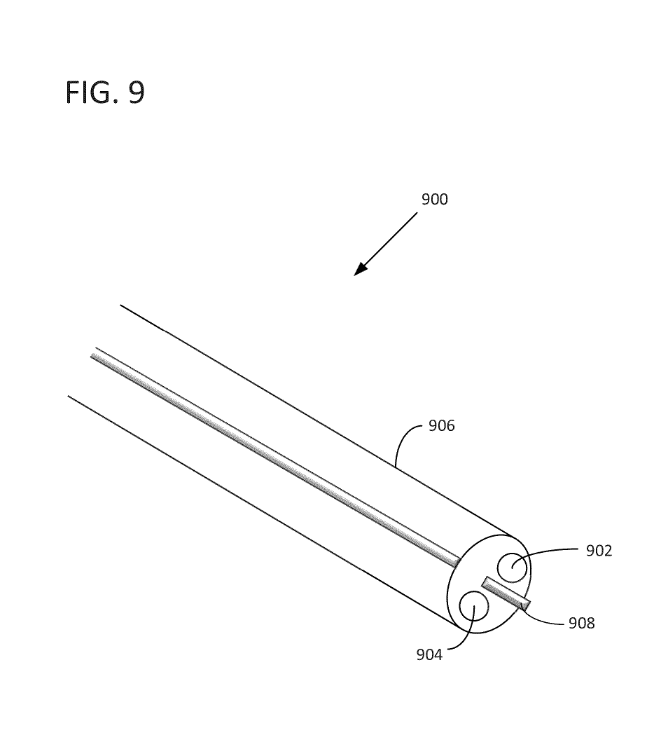

With reference to FIG. 9, an endoscope 900 includes a tube 906 that contains a coherent fiber bundle that terminates at a probe tip 908. A lens can be provided to image a target region into the coherent fiber bundle so that spectral imaging and image processing can be performed at a remote location. Fibers 902, 904 can be coupled to visible or other radiation sources for target imaging at visible wavelengths, or to provide excitation radiation suitable to produce target fluorescence. The endoscope 900 can be rigid or flexible, the probe end 908 does not contact the target in operation.

### Example 8

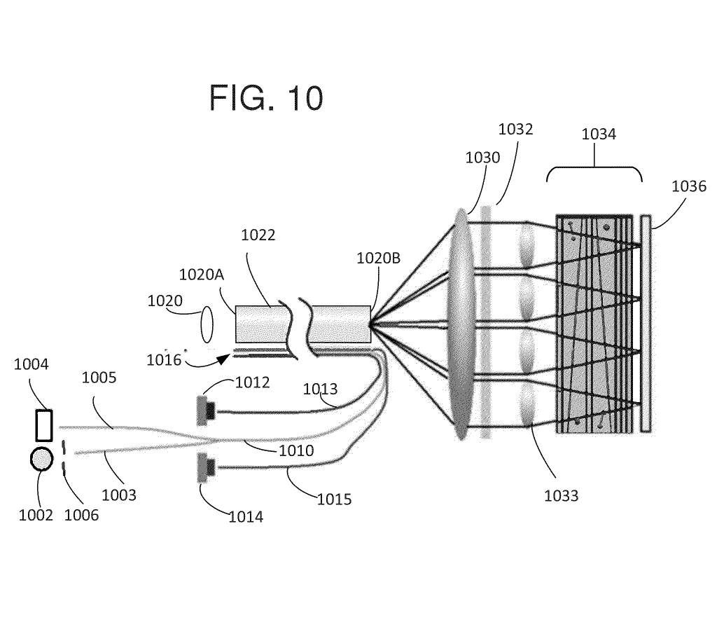

Referring to FIG. 10, an endoscope system includes a quartz halogen lamp 1002 situated to direct optical radiation to a fiber 1003 through a shutter 1006. A xenon flash lamp 1004 is situated to direct optical radiation to a fiber 1005, and combined quartz halogen and xenon radiation are coupled into a single fiber 1010. LEDS/laser diodes 1012, 1014 couple excitation optical beams into fibers 1013, 1015, respectively and a combined fiber assembly 1016 delivers LED/laser and other beams to a target region. A coherent fiber bundle 1022 delivers an image produced by a lens 1020 at a distal end 1022A to a proximal end 1022B. A collimating lens 1030 directs the received image (based on fluorescence, excitation, visible beams) through an excitation blocking filter 1032 that attenuates excitation radiation. Optical beams from the lamps 1002, 1004 can be eliminated with the shutter 1006 or suitable timing of xenon lamp excitation. A lenslet array 1033 directs the filtered image beam to a birefringent spectral analyzer 1034 and to an array detector 1036. The image at the array detector 1036 is processed to produce an image having a plurality of spectral slices that can be further processed to evaluate particular specimen conditions.

### Example 9

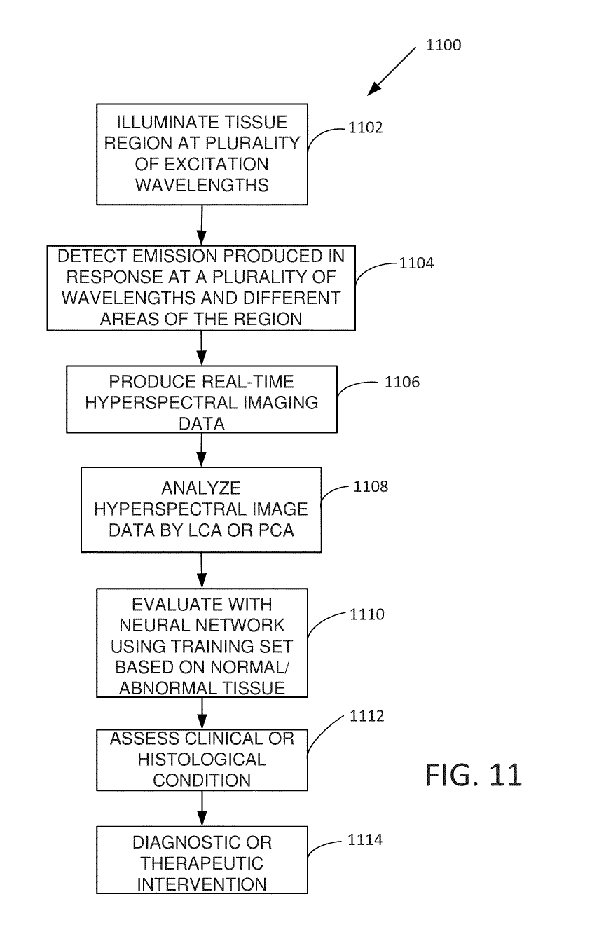

Referring to FIG. 11, a method 1100 includes directing one or more excitation beams to a target tissue at 1102 so as to produce fluorescence associated with a particular cell type or cellular condition in the tissue. At 1104, fluorescence is detected at one or more wavelengths (generally over substantially all of the emission bandwidth) so that real-time hyperspectral images are produced at 1106. Principal component analysis or linear component analysis are used to process one or more images at 1108. At 1110, a neural network evaluates the processed images to identify features of interest and/or to characterize tissue regions. Clinical or histological assessments are conducted at 1112, and diagnosis or therapy is provided at 1114. In some cases, diseased or suspicious tissues are recognized based on fluorescence power produced as a function of excitation wavelength or power.

### Example 10

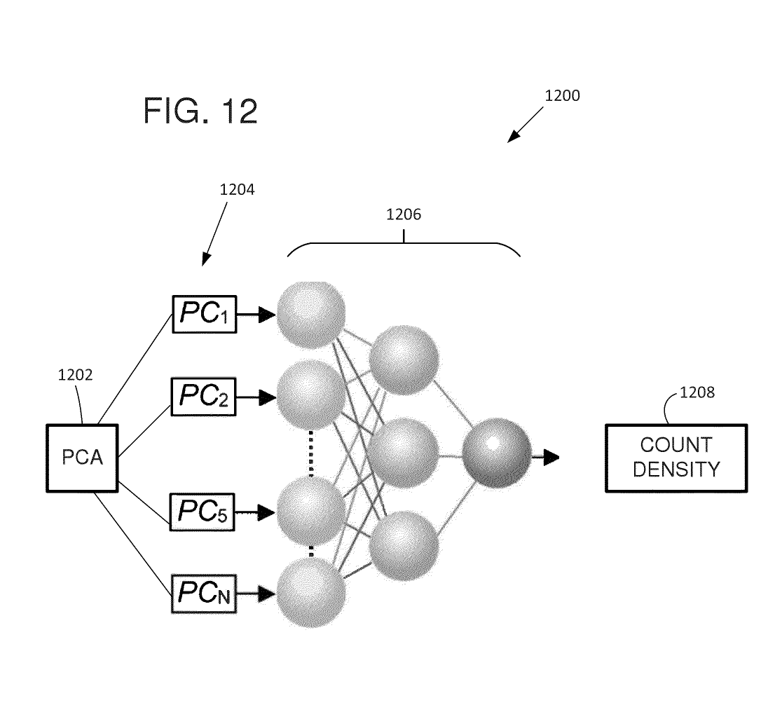

FIG. 12 illustrates a method 1200 of processing of hyperspectral images for tissue evaluation. At 1202, image data is processed by PCA to identify a plurality of principal components 1204 that are provided to a neural network 1206. Based on the output of the neural network 1206, a count density of suspicious cells or other clinically useful tissue characteristic is obtained at 1208, and can be combined with conventional tissue images.

### Example 11

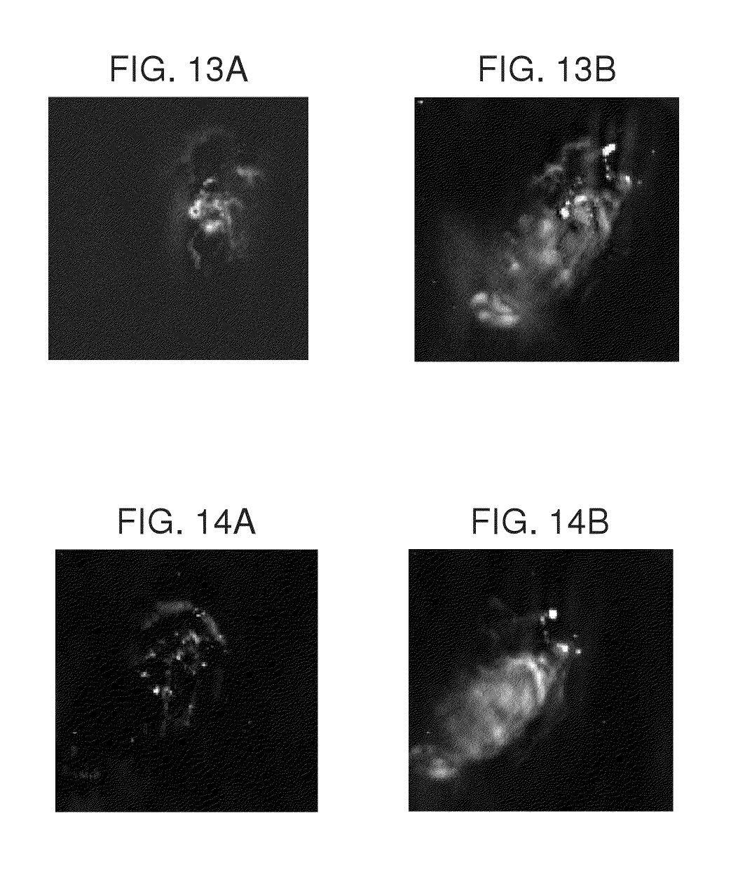

The disclosed methods and apparatus were used with a cell phantom for demonstration purposes. The cell phantom consisted of fluorescent polystyrene beads with a diameter of 2 [UNITS]. These beads were used to simulate disease related to increased fluorescence from flavin adenine dinucleotide (FAD). A 407 nm (blue) laser diode was used as excitation source for the beads and produced a green (˜500 nm) fluorescent signature. Linear component analysis (LCA) was used to isolate the microsphere's spectrum from that of the background tissue's autofluorescence at each pixel within the scene. First, LCA was performed on a high concentration microsphere image to verify that the LCA algorithm was properly extracting the microspheres from the background. These results are provided in shown in FIGS. 13A-13B for the microspheres and background, respectively. From these results, it is apparent that the relatively dim background spectrum is successfully extracted from that of the bright microspheres. LCA was then performed on images acquired with a much lower microsphere concentration, such that the microspheres were difficult to discern with the unaided eye when illuminated by the 407 nm excitation source. These resulting images for the microspheres and background are shown in FIGS. 14A-14B, respectively.

### Example 12

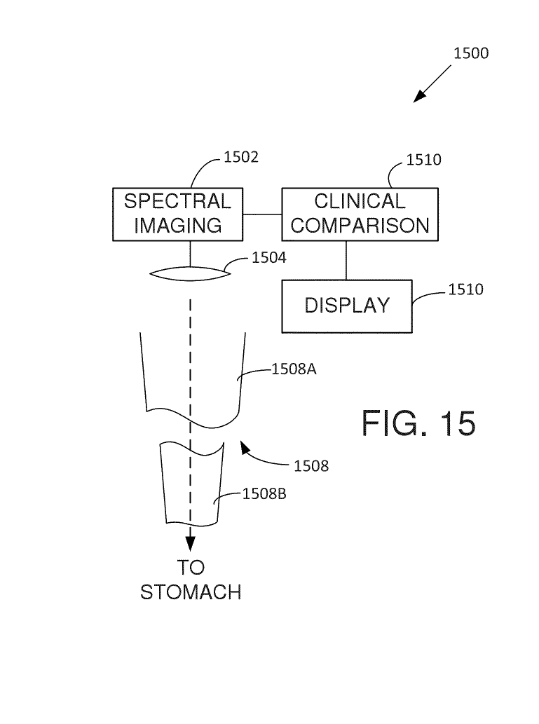

Unlike conventional methods, the disclosed methods and apparatus permit simple, contact free assessment of the esophagus and other structures. While contact with an endoscope and tissue may occur, such contact is incidental to measurement. As shown schematically in FIG. 15, a system 1500 includes a spectral imager 1502 that is coupled to a clinical processor 1504 that can assess tissue based on the spectral images. Assessments can be provided to a clinician as one or more images on a display device 1510. The spectral imager 1502 is configured to receive an image from a lens 1504 that is situated along an axis 1505 that extends substantially along an esophagus 1508 so that an upper section 1508A and a lower section 1508B are imaged in a single image. A clinician is thus able to view down the axis, permitting real time assessment of large areas of the esophagus as well as ready determination of tissue abnormalities locations. Such images are especially important as tissue abnormalities (the presence of eosinophils) tend to cluster so that inspection of a small are may miss significant tissue abnormalities. In invasive assessments based on biopsies, tissue samples at four or more locations can be required. As disclosed herein, a single snapshot spectral image permits assessment of the esophagus over a substantial length, and a few such images are sufficient for complete evaluation.

Axial imaging also permits simple estimation of the location of abnormalities, which can be important in diagnosis. Eosinophils near the stomach tend to be associated with reflux, while eosinophils in upper portions can indicate allergic reactions. Axial images inform the clinician of eosinophil location and density, simplifying diagnosis.

### Example 13

In one implementation, a probe includes a flexible, water-proof, fiber image conduit that can be inserted into a patient's esophagus. The probe is configured to measure two sets of continuous (500-700 nm) emission spectra when illuminated sequentially by two excitation sources. A wide field objective lens images the esophagus onto the fiber conduit, and separate fibers can be used for tissue illumination. Sources can include a 405 nm and 450 nm optically-coupled light emitting diode (for excitation) and a xenon white light flash lamp to allow calculation of the intrinsic AF spectrum at both excitations. Excitation at other or additional wavelengths can also be used. A microcontroller is configured to time-sequentially pulse the LEDs and the xenon flash lamp when acquiring data and synchronize these illumination events to camera exposures. Lastly, a shuttered tungsten-halogen lamp enables continuous imaging when not acquiring AF data. Assuming a 2048×2048 pixel element CCD camera, the spectrometer can achieve a 256×256 pixel spatial resolution datacube with 38 spectral channels (slices) spanning 500-700 nm (Δλ˜5.2 nm).

### Example 14

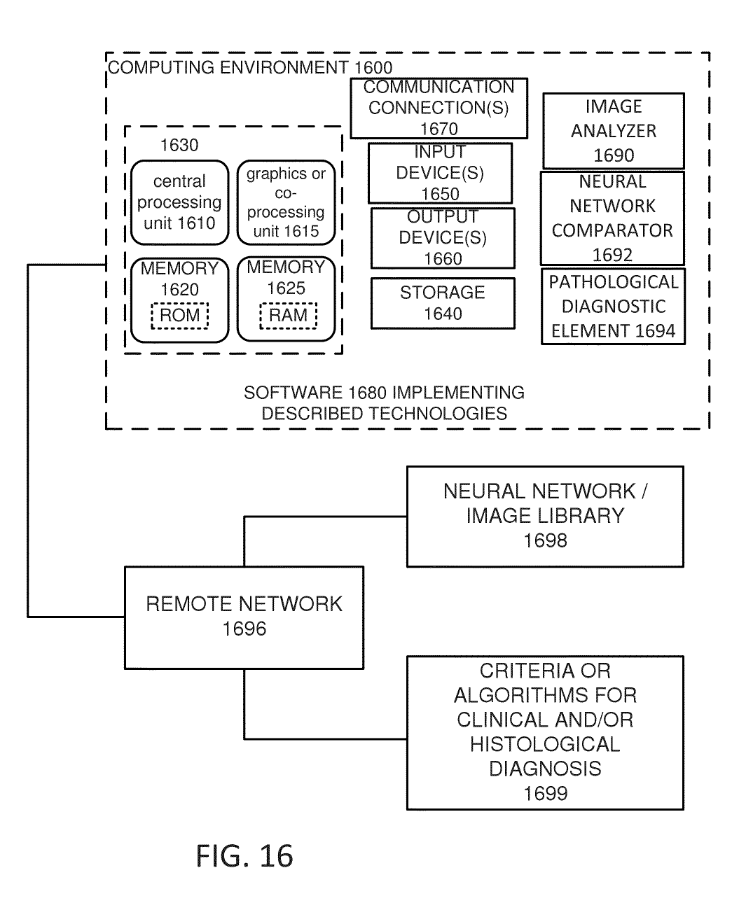

FIG. 16 depicts a generalized example of a suitable computing environment 1600 in which the described innovations may be implemented. The computing environment 1600 is not intended to suggest any limitation as to scope of use or functionality, as the innovations may be implemented in diverse general-purpose or special-purpose computing systems. For example, the computing environment 300 can be any of a variety of computing devices (e.g., desktop computer, laptop computer, server computer, tablet computer, mobile device, etc.).

With reference to FIG. 16, the computing environment 1600 includes one or more processing units 1610, 1615 and memory 1620, 1625. In FIG. 16, this basic configuration 1630 is included within a dashed line. The processing units 1610, 1615 execute computer-executable instructions. A processing unit can be a general-purpose central processing unit (CPU), processor in an application-specific integrated circuit (ASIC) or any other type of processor. In a multi-processing system, multiple processing units execute computer-executable instructions to increase processing power. For example, FIG. 3 shows a central processing unit 1610 as well as a graphics processing unit or co-processing unit 1615. The tangible memory 1620, 1625 may be volatile memory (e.g., registers, cache, RAM), non-volatile memory (e.g., ROM, EEPROM, flash memory, etc.), or some combination of the two, accessible by the processing unit(s). The memory 1620, 1625 stores software 1680 implementing one or more innovations described herein, in the form of computer-executable instructions suitable for execution by the processing unit(s). In some examples, computer-executable instructions and associated data for image analysis, neural network processing, and diagnosis are stored in memory portions 1690, 1692, 1694, respectively.

A computing system may have additional features. For example, the computing environment 1600 can include storage 1640, one or more input devices 1650, one or more output devices 1660, and one or more communication connections 1670. An interconnection mechanism (not shown) such as a bus, controller, or network interconnects the components of the computing environment 1600. Typically, operating system software (not shown) provides an operating environment for other software executing in the computing environment 1600, and coordinates activities of the components of the computing environment 1600.

The tangible storage 1640 may be removable or non-removable, and includes magnetic disks, magnetic tapes or cassettes, CD-ROMs, DVDs, or any other medium which can be used to store information in a non-transitory way and which can be accessed within the computing environment 1600. The storage 1640 stores instructions for the software 1680 implementing one or more innovations described herein.

The input device(s) 1650 may be a touch input device such as a keyboard, mouse, pen, or trackball, a voice input device, a scanning device, or another device that provides input to the computing environment 1600. For video encoding, the input device(s) 1650 may be a camera, video card, TV tuner card, or similar device that accepts video input in analog or digital form, or a CD-ROM or CD-RW that reads video samples into the computing environment 1600. The output device(s) 1660 may be a display, printer, speaker, CD-writer, or another device that provides output from the computing environment 1600.

The communication connection(s) 1670 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

Any of the disclosed methods can be implemented as computer-executable instructions stored on one or more computer-readable storage media (e.g., one or more optical media discs, volatile memory components (such as DRAM or SRAM), or nonvolatile memory components (such as flash memory or hard drives)) and executed on a computer (e.g., any commercially available computer, including smart phones or other mobile devices that include computing hardware). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C++, Java, Perl, JavaScript, Adobe Flash, or any other suitable programming language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means. As shown in FIG. 16, a remote network 1696 is coupled to a neural network and image library 1698 as well as a library containing diagnostic criteria and algorithms.

### Example 15

Autofluorescence images can be compared to histologic mapping to assess spatial correlation. Eosinophil count/high power field can be correlated to fluorescence spectra by training a neural network. A 1:1 mapping can be generated between histological findings and measured data using a grid superimposed on specimen image. A training dataset can be created in which the measured intrinsic fluorescence can be directly related to the abundance of eosinophils per HPF. This can generate a truth dataset that can be used to train a feed-forward neural network algorithm to identify the abundance of eosinophils against background fluorescence. A basic feed-forward neural network including several interconnected neurons accept inputs at an input layer. These inputs can consist of at least the first 10 principle components (PCN) from a measured intrinsic fluorescence for each excitation source (e.g., 20 total inputs with 10 components from 405 nm and 450 nm). These components are transmitted from the input through one or more hidden layers, the number of which can be determined empirically based on the network's performance, and the biasing of which is established during network training. Thus, these training data allow the network to establish a statistical correlation between the measured signal and the output eosinophil concentration for subsequent testing on a new set of data.

### Example 16

In one example, the hyperspectral spectrometer can be based on an existing Snapshot Hyperspectral Imaging Fourier Transform (SHIFT) spectrometer such as disclosed in Kudenov, U.S. Patent Application Publication 20120268745. The SHIFT spectrometer benefits from the multiplex advantage when detector-noise is limited (i.e., photon-starved); (2) is extremely compact (currently 15×15×6 mm³ without the camera); (3) offers high spectral and spatial resolution with continuously sampled spectra, with real-time output; and (4) can realize tunable spectral resolution. A Fourier transformation of this cube, along the OPD axis, allows the spectrum to be extracted for all spatial locations within a single snapshot. Additionally, post-processing is highly parallel. A 5 frame-per-second reconstruction rate on a 220×220×100 pixel interferogram cube using highly parallel graphics processing unit-based code has been demonstrated.

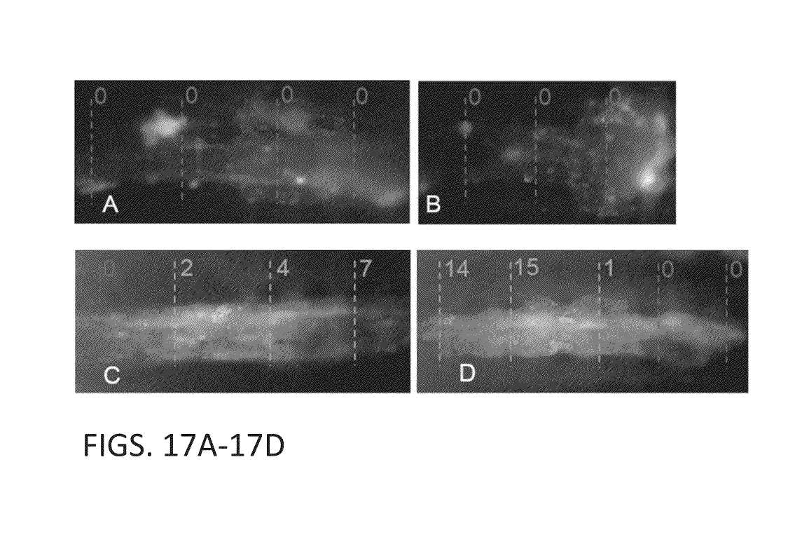

A SHIFT spectrometer was configured for hyperspectral imaging experiments on freshly resected murine esophagi to image autofluorescence (AF) signatures of EoE and normal esophagi in pathogen-free BALB/c mice. AF spectra (fluorescence intensity I(x,y,λ), wherein x, y are spatial coordinates, and λ emission wavelength) were obtained sequentially under 405 nm (I₄₀₅(x,y,λ)) and 450 nm I₄₅₀(x,y,λ)) laser excitation light to exploit uniqueness generated by the target tissue's continuous emission spectra at two excitation wavelengths. Esophageal white light reflectance and spectral calibration images were also obtained to calculate intrinsic fluorescence. A neural network algorithm was not used. Measurements from 500-530 nm were spectrally band-integrated and the ratio R=I₄₀₅/I₄₅₀ was calculated. Peanut-extract (for EoE; n=4) and normal saline (control; n=2) were administered. Mice (n=6) were sacrificed, esophagi resected, cut longitudinally, and the mucosal surface imaged within 15 minutes by the SHIFT spectrometer, ex vivo on top of a non-fluorescent grid with 1 mm² intersections. After the images were acquired, the tissue was stained in locations coincident with the grid to guide histology, thus preventing tissue contraction from skewing the histology's image registration. An image of the middle and distal ends of one control esophagus is provided in FIG. 17A and FIG. 17B, respectively, showing significant differences in the AF ratio (R) when compared to the middle and distal ends of an EoE esophagus shown in FIG. 17C and FIG. 17D), respectively. The presence of eosinophils in lung biopsies, in addition to esophageal tissue, was used to confirm EoE (3 out of 4 peanut extract mice developed EoE). Histological specimens (tissue slices) were obtained, and the specimens were processed, stained, and examined for eosinophils at 40× magnification. The number of eosinophils per HPF was counted in 3 unique regions of each slice, the average of which is presented alongside the dashed overlays of FIGS. 17A-17D. While one false negative exists, there is good correlation between the number of eosinophils/HPF and the areas of increased fluorescence ratio (R) in the EoE tissue when compared to the control; a consistent feature across all preliminary data. Continuous (i.e., not band-integrated) intrinsic AF spectra can be used as input into neural network to reduce false signatures. Preliminary data supports the hypothesis that EoE's autofluorescence contains potentially diagnostic spectral characteristics. Datacube reconstruction (determination of I(x,y,λ)) and other analysis can be performed with computer-executable instructions provided in MATLAB computational software.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope of these claims.

We claim:

1. A system for real-time in vivo imaging of a tissue sample region containing at least one autofluorescent cell, comprising:
an excitation source configured to deliver excitation radiation to the tissue sample region at one or more excitation wavelengths;
a snapshot spectral imager configured to receive optical radiation emitted in response to the excitation radiation from the tissue sample region from at least one autofluorescent cell; and
an image processor configured to detect a target feature in the tissue sample region based on the spectral images.

2. The system of claim 1, wherein the target feature is an autofluorescent cell.

3. The system of claim 1, wherein the autofluorescent cell is an eosinophil.

4. The system of claim 1, wherein the image processor is configured to determine an estimate of a number of target features per target area in the tissue sample region.

5. The system of claim 4, wherein the image processor is configured to provide a processed image associated with detected target features and the estimate of the target features per target area in the tissue sample.

6. The system of claim 1, wherein spectral imager is situated within an endoscope configured for insertion into a body lumen.

7. The system of claim 1, wherein the spectral imager is configured to produce an esophageal image corresponding to a view along an esophageal axis, and wherein the target feature is an eosinophil.

8. The system of claim 1, wherein the excitation source is configured to deliver excitation radiation to the tissue sample region at a first excitation wavelength and a second excitation wavelength, and the image processor is configured to detect the target feature based on ratios of received emitted optical power associated with the first excitation wavelength and the second excitation wavelength optical radiation at a plurality of emission wavelengths.

9. A method for analyzing a tissue sample region containing at least one autofluorescent cell, comprising:
irradiating the region at a plurality of excitation wavelengths;
detecting emitted optical radiation from the at least one autofluorescent cell at a plurality of emission wavelengths generated in response to the irradiation; and
identifying a location of the at least one autofluorescent cell based on the detected optical radiation at the plurality of emission wavelengths.

10. The method of claim 9, wherein the emitted optical radiation is detected so as to form corresponding spectral images, and the location of the at least one autofluorescent cell is identified based on the spectral images.

11. The method of claim 10, wherein the location of the at least one autofluorescent cell is identified based on ratios of received emitted optical radiation associated with the first excitation wavelength and the second excitation wavelength at the plurality of emission wavelengths.

12. The method of claim 9, wherein the emitted optical radiation from the at least one autofluorescent cell is detected by snapshot imaging so as to form spectral images based on emitted optical radiation associated with the first and second excitation wavelengths.

13. The method of claim 12, wherein the target region is a portion of an esophagus, and the snapshot images are images viewing along an axis of the esophagus.

14. The method of claim 13, further comprising determining a density of identifying a density of a plurality of autofluorescent cells at a plurality of locations in the tissue sample region.

15. The method of claim 14, further comprising displaying an image of the target region that includes an indication of a clinical level associated with the density of the plurality of autofluorescent cells.

16. The method of claim 15, wherein the indication is associated with a coloration of the displayed image or numerical values applied to the displayed image.

17. The method of claim 15, wherein the clinical level is dependent on axial location in the esophagus.

18. A computer readable storage medium, having stored data representing computer executable instructions for a method comprising:
processing first and second spectral images of a tissue sample region based on fluorescence emitted from the tissue sample region in response to excitation optical radiation at a first wavelength and a second wavelength, respectively; and
identifying at least one target cell or at least one background cell based on the processed first and second spectral images.

19. The computer readable storage media claim 18, further comprising:
determining a target cell relative abundance based on identification of a plurality of target cells;
producing an output image based on the processed first and second spectral images that visually distinguishes the target cells from a background tissue; and
providing a display of a clinical condition at a plurality of locations in the output image based on the relative abundance, the clinical condition selected from the group consisting of eosinophilia, lymphocytosis, leukopenia, and platelet deficiency.

20. The computer readable storage medium of claim 18, wherein the first and second spectral images of a tissue sample region are processed to obtain ratios of fluorescence emitted in response to excitation at the first wavelength and the second wavelength, and the at least one target cell is identified based on the ratios.
